(19) United States
(12) Patent Application Publication
(10) Pub. No.: US 2025/0017607 A1
(43) Pub. Date: Jan. 16, 2025

(54) LITHOTRIPSY DEVICE, IN PARTICULAR INTRACORPOREAL LITHOTRIPSY DEVICE, FOR BREAKING UP CALCULI, IMPACT ELEMENT, RETROFITTING SET FOR RETROFITTING AN EXISTING LITHOTRIPSY DEVICE, AND METHOD FOR OPERATING A LITHOTRIPSY DEVICE

(71) Applicant: KARL STORZ SE & Co. KG, Tuttlingen (DE)

(72) Inventors: Florian HUBER, Tuttlingen (DE); Bernhard GLÖGGLER, Tuttlingen (DE)

(21) Appl. No.: 18/709,858

(22) PCT Filed: Nov. 22, 2022
(86) PCT No.: PCT/EP2022/082721

§ 371 Date: May 14, 2024

(30) Foreign Application Priority Data
Nov. 24, 2021 (DE) 10 2021 130 793.7

(51) Int. Cl. A61B 17/22; A61B 17/225

(52) U.S. Cl. CPC A61B 17/22012; A61B 17/225; A61B 2017/22015

(57) ABSTRACT

An intracorporeal lithotripsy device, impact body, retrofit kit and method fragment body stones. The lithotripsy device has a carrier unit, a sonotrode, connectable to the carrier unit at the distal end and a longitudinal center axis, and an impact body for mechanical impact excitation of the sonotrode. The impact body has a longitudinal direction and is connected to a clamped elastic element and the lithotripsy device has a vibration excitation device for vibration excitation of the sonotrode and of the impact body, and the free end of the impact body is directed towards a first excitation surface and can be pressed against the first excitation surface by means of the clamped elastic element upon vibration excitation. The impact body is arranged so that radial vibration of the sonotrode can be excited by an oblique strike of the impact body with its free end onto the first excitation surface.

### CROSS REFERENCE TO RELATED APPLICATIONS

This application is a United States National Phase Application of International Application PCT/EP2022/082721, filed Nov. 22, 2022, and claims the benefit of priority under 35 U.S.C. § 119 of German Application 10 2021 130 793.7, filed Nov. 24, 2021, the entire contents of which are incorporated herein by reference.

### TECHNICAL FIELD

The present invention pertains to a lithotripsy device, in particular an intracorporeal lithotripsy device, for fragmenting body stones, wherein the lithotripsy device has a carrier unit, a sonotrode that is connectable to the carrier unit at the distal end and has a longitudinal center axis, and at least one impact body for mechanical impact excitation of the sonotrode, wherein the at least one impact body has a longitudinal direction, a proximal end and a distal end and is connected at its proximal end or at its distal end to a clamped elastic element, so that the at least one impact body has a free end, and the lithotripsy device has a vibration excitation device for vibration excitation of the sonotrode and of the at least one impact body, and the free end of the at least one impact body is directed towards a first excitation surface and can be pressed against the first excitation surface by means of the clamped elastic element upon vibration excitation, wherein the excitation surface is connected directly or indirectly to the sonotrode.

### BACKGROUND

Lithotripsy is a well-known method for fragmenting body stones, which form as so called concretions in body organs, for example in the bladder or kidneys, due to the crystallization of salts. If the body stones are too large for natural passage and cause discomfort, they must be crushed using a lithotripter so that the crushed stones can be removed by natural excretion and/or using a suction/rinsing pump.

Such body stones are often not homogeneous in structure, but have different components, layers and/or strengths. Thus, a body stone which has a soft concretion on the outside and a hard stone core on the inside requires a higher fragmentation force of the impact waves emitted by the lithotripter with increasing fragmentation.

For these reasons, purely ultrasound-based lithotripters have been further developed in recent years to improve stone fragmentation performance. For this purpose, intermittent ballistic shock wave energy is usually applied in addition to the constant ultrasonic energy. For example, a projectile can be accelerated in an acceleration tube by means of a pneumatic drive and hit a horn and/or the sonotrode head. The disadvantage here is that the striking is only effected directly or indirectly to the sonotrode head from an axial direction and, in the case of an intracorporeal lithotripter, the distal end opposite the sonotrode head is only driven further into the body stone in this one direction.

U.S. Pat. No. 9,421,023 B2 discloses a device for the transmission of ultrasonic vibrations, with which the ultrasonic waveguide is received on the proximal side in a waveguide fitting, and a first shock-pulsing mass, followed by a compression spring, and a second shock-pulsing mass are arranged coaxially around the ultrasonic waveguide on the distal side of the waveguide fitting. The second shock-pulsating mass is delimited on the distal side by a hitting surface, onto which the second shock-pulsating mass strikes when excited by the ultrasonic vibration. Due to the shaping of the two impact-pulsing masses, a non-uniform impact can occur in which the second impact-pulsing mass strikes an edge of the hitting surface obliquely. This stochastic, oblique impact of the second shock-pulsing mass has a relatively minor effect on the vibration of the sonotrode and cannot be specifically adjusted. Furthermore, the oblique position of the second impact-pulsing mass is limited in design due to the coaxial arrangement around the ultrasonic waveguide and only a single striking mass can strike the hitting surface. In addition, as a result of this limitation, the device cannot be subsequently integrated into ultrasonic transducers and sonotrodes already on the market.

### SUMMARY

An object of the invention is to improve the prior art. The object is achieved by a lithotripsy device, in particular an intracorporeal lithotripsy device, for fragmenting body stones, wherein the lithotripsy device has a carrier unit, a sonotrode that is connectable to the carrier unit at the distal end and has a longitudinal center axis, and at least one impact body for mechanical impact excitation of the sonotrode, wherein the at least one impact body has a longitudinal direction, a proximal end and a distal end and is connected at its proximal end or at its distal end to a clamped elastic element, so that the at least one impact body has a free end, and the lithotripsy device has a vibration excitation device for vibration excitation of the sonotrode and of the at least one impact body, and the free end of the at least one impact body is directed towards a first excitation surface and can be pressed against the first excitation surface by means of the clamped elastic element upon vibration excitation, wherein the excitation surface is connected directly or indirectly to the sonotrode, and the at least one impact body is arranged with its longitudinal direction in a direction deviating from the longitudinal center axis of the sonotrode and deviating from a transverse axis of the sonotrode, so that radial vibration of the sonotrode can be excited by an oblique strike of the at least one impact body with its free end onto the first excitation surface.

Thus, a lithotripsy device is provided with a striking unit acting non-coaxially and non-perpendicularly with respect to the sonotrode, the striking unit consisting of at least one impact body as a striking mass and the connected pretensioned elastic element, wherein a defined oblique striking excitation is realized as a result of the spatial arrangement of the at least one impact body as a result of the oscillating impact body during operation.

Due to the oblique strike of the at least one impact body on the first excitation surface, a reproducible and intensive radial vibration of the sonotrode is excited. In addition to the usual longitudinal vibration excitation of the sonotrode, radial excitation of the sonotrode is also achieved. In intracorporeal lithotripters, a digging in and/or jamming of the distal sonotrode tip in the body stone in the direction of the longitudinal center axis of the sonotrode is prevented, and the specifically introduced radial vibration causes a drilling effect upon the fragmentation of the body stone. This increases the removal rate and fragmentation performance.

In addition, the striking frequency for mechanical impact excitation can be specifically adjusted by selecting the mass of the impact body and the elastic element as a spring.

It is particularly advantageous that the excitation surface not only represents the striking surface for the oscillating impact body, but can itself be excited with a vibration, for example an ultrasonic vibration, by means of the vibration excitation device and thus the at least one impact body can hit the vibrating excitation surface. The vibration of the excitation surface repulses the striking mass of the at least one impact body and thus tensions the elastic element connected to the impact body as a spring. The spring stores the absorbed energy and returns it to the impact body, as a result of which a renewed impact on the vibrating excitation surface is exerted. These processes are repeated continuously as long as vibration excitation is present.

It is particularly advantageous that the spatial alignment of the impact body with its longitudinal direction and thus its oblique position between a direction of the transverse axis transverse to the longitudinal center axis of the sonotrode and the longitudinal center axis of the sonotrode can be freely selected and thus the degree of radial excitation of the sonotrode can be freely adjusted.

An essential concept of the invention is based on the fact that both the at least one impact body and the excitation surface are not permanently installed coaxially to the longitudinal center axis of the sonotrode, but are arranged in a spatially free and/or oblique manner. As a result of the fact that the at least one impact body is arranged with its longitudinal direction independent of the direction of the longitudinal center axis of the sonotrode and the excitation surface in the direction of the impact excitation is not directly or indirectly in contact with the region of the cross-section of the sonotrode and is thus arranged in the direction of the longitudinal center axis of the sonotrode, the oblique impact of the at least one impact body can be specifically adjusted in its strength and alignment on the first excitation surface and thus a defined radial vibration of the sonotrode can be used to improve the fragmentation performance.

The following terminology is explained:

A “lithotripsy device” (also known as a “lithotripter”) is in particular a device for fragmenting body stones using impact waves. A lithotripsy device is understood to mean in particular various components, structural and/or functional elements of a lithotripter. The lithotripsy device can completely or partially form a lithotripter. A lithotripsy device can in particular be an intracorporeal or extracorporeal lithotripsy device. In the case of an intracorporeal lithotripsy device, this can also have a rinsing/suction pump. The lithotripsy device can be designed as a hand-held device and/or have an endoscope or be inserted into an endoscope. The lithotripsy device is in particular autoclavable and comprises, for example, instrument steel and/or plastics. The lithotripsy device can have further components, such as a control and/or supply unit, or these are assigned to the lithotripsy device.

“Body stones” (also known as “concretions”) are understood to mean in particular all stones in a human or animal body that are formed from salts through crystallization. Body stones can be, for example, gallstones, urinary stones, kidney stones and/or salivary stones.

A “carrier unit” is in particular a hand and/or holding part of the lithotripsy device. In particular, the carrier unit can be a handle for manual and/or automated operation and/or connection of the lithotripsy device. The carrier unit can also be arranged, connected and/or automatically guided at a distal end of a robot arm. In particular, the carrier unit has a housing.

A “sonotrode” is in particular a component that is itself set into vibration and/or resonant vibration by the action and/or introduction of mechanical vibrations. The sonotrode is excited in a vibration, in particular longitudinal vibration, in particular by means of the vibration excitation device, for example with an ultrasonic vibration exciter. In addition, the sonotrode is excited in particular in a radial vibration by means of the impact body. The sonotrode is designed in particular as a waveguide for the vibration waves generated by the vibration excitation device and for the impact waves (pulses) of the impact body. In particular, the sonotrode is connected to the ultrasonic vibration exciter, the ultrasonic transducer and/or the horn. For example, the sonotrode is screwed into the distal end of the horn. In particular, the sonotrode has a sonotrode head at its proximal end for receiving, forwarding and/or focusing ultrasonic waves and a sonotrode tip at its distal end for directly and/or indirectly applying and/or contacting body stones. In particular, the sonotrode is shaped in such a way that it optimally introduces the vibration waves, impact waves and/or the ultrasonic vibration at its distal end into the body, the body region to be treated and/or directly onto the body stone to be fragmented. In the case of ultrasonic excitation, the sonotrode operates in particular in the ultrasonic range with a frequency range from 20 kHz to 90 kHz, preferably from 20 kHz to 34 kHz. The sonotrode is made of steel, titanium, aluminum and/or carbon. A sonotrode is, in particular, a probe that is, for example, rod-shaped, tubular and/or hose-shaped. The sonotrode can be made in one piece or in multiple parts. In particular, the sonotrode has a diameter in the range from 0.5 mm to 4.5 mm, in particular from 0.8 mm to 3.8 mm.

The “longitudinal center axis” is in particular the axis of the sonotrode that corresponds to the direction of its greatest extension. A “transverse axis” is in particular the axis of the sonotrode that is perpendicular to the longitudinal center axis of the sonotrode.

“Distal side” and “distal” are understood to mean an arrangement and/or end that is close to the body and therefore remote from the user. Accordingly, “proximal side” or “proximal” is understood to mean an arrangement close to the user and thus remote from the body or a corresponding end.

A “vibration excitation device” is in particular any device that induces vibration excitation of the sonotrode. In particular, the vibration excitation device excites a regular and/or constant vibration. For example, a laser or pneumatic energy source can be used for this purpose. Preferably, the vibration excitation device has an ultrasonic vibration exciter.

An “ultrasonic vibration exciter” (also called a “vibration exciter”) is in particular a component of an ultrasonic transducer and/or handpiece of a lithotripsy device, which converts a supplied alternating voltage with a specific frequency into a mechanical vibration frequency. The ultrasonic vibration exciter is in particular an electromechanical transducer utilizing the piezoelectric effect. Due to the application of an alternating electrical voltage generated by an ultrasonic generator, a mechanical vibration is generated in particular as a result of deformation of the ultrasonic vibration exciter. In particular, the ultrasonic vibration exciter has one or more piezo elements. Preferably, the ultrasonic vibration exciter has at least two piezo elements, wherein an electrical conductor, for example a copper disk, can be arranged between the piezo elements. The ultrasonic vibration exciter and/or the ultrasonic transducer can in particular have a horn.

A “horn” is in particular a component that is arranged between the vibration exciter and/or a piezo element and the sonotrode. The horn is used in particular to transmit, forward and/or align the ultrasonic waves generated by the vibration exciter to the sonotrode. For this purpose, the horn can taper in a transmission direction and transmit the ultrasonic waves directly or indirectly to a sonotrode head. The horn can also be used to fasten the sonotrode. At the same time, the horn serves to mechanically hold the piezo element or piezo elements on both sides, in particular together with a counter bearing.

An “impact body” (also known as a striking body) is understood to mean in particular a body, a component and/or an assembly that can be struck obliquely on an excitation surface. In particular, the impact body is free of a coaxial arrangement to the longitudinal center axis of the sonotrode. In particular, the impact body has a mass that oscillates during the operation of the lithotripsy device, as a result of which the impact body moves back and forth along its longitudinal direction and/or a striking direction. In principle, the impact body can have any shape and does not necessarily have to be designed to be rotationally symmetrical. Preferably, the impact body has its largest longitudinal dimension in the striking direction. Instead of an elongated body, the impact body can also be designed as a ball in an elongated cage, for example, so that only a limited contact point is effective upon striking the excitation surface. The impact body can also be tapered in the striking direction, for example, so that, as a result of the smaller surface area of the tapered tip of the impact body, higher surface pressure is present upon striking.

In addition to the shape, the mass and size of the impact body can also be freely selectable. These properties also depend on the arrangement of the impact body within the lithotripsy device. If the impact body is arranged on the horn, for example, it may be advantageous that the impact body is smaller than the diameter of the horn and, in particular, has a mass of one tenth of the horn mass. If, on the other hand, the impact body is arranged both on the horn and on the sonotrode, for example at their transition region, the sonotrode and the horn represent a large effective mass and the striking mass of the impact body can optimally correspond to the mass of the horn. In general, it should be taken into account that the mass of the impact body should not be too large, since as a result of the interaction of the mass of the impact body and the elastic element as a spring, the movement and thus the striking of the impact body becomes relatively sluggish.

An “elastic element” is a component that can be elastically deformed during the operation of the lithotripsy device. The elastic deformation of the elastic element is in particular bending, torsion, elongation and/or compression. In particular, the elastic element is compressed and/or expanded in the striking direction. An elastic element can be a spring in particular. An elastic element, for example, is a helical spring, which consists of a wire made of metal and/or plastics wound in a helical shape. The elastic element can be a helical tension and compression spring. However, an elastic element can also be an elastic plastics body free of a helical shape. For example, the elastic element is designed as a cylinder made of resilient plastics, such as an elastomer. Preferably, the elastic element is designed to be stable in such a way that it guides the mass of the impact body. The spring can be connected to one end and/or one surface of the impact body in any manner, and thus by a form-fit, force-fit and/or material-fit connection. For example, the impact body has a recess in which the spring engages. The recess can, for example, be a spiral-shaped groove in the impact body into which the coil of the spring engages.

“Elasticity tension” characterizes in particular a property of the elastic element to change its shape when a force is applied and to return to its original shape when the applied force is removed. Elasticity tension is in particular a spring tension (also known as spring stiffness), which indicates the ratio of the force acting on the elastic element as a spring to the resulting deflection of the spring.

The elastic element is connected to the impact body at its end in alignment in the striking direction and is clamped at its opposite end, in particular by a limiting element. The limiting element can be a wall of a bracket or the horn, for example. A limiting element can also be a receiving element. A receiving element can, for example, be a hollow cylinder in which the end of a rod-shaped and/or helical-shaped elastic element is received and thereby both guided and clamped, wherein the end of the elastic element abuts against an end wall of the hollow cylinder or a wall of a holder or the horn against the striking direction.

The impact body and the connected elastic element thus form a mass-spring system, with which the resiliently mounted impact body can perform mechanical vibrations excited by the vibration excitation device. Depending on the properties of this mass-spring system to be adjusted, the impact body and the elastic material are made of different materials, wherein both a relatively high strength and high elasticity of the spring are required for a directionally accurate impact of the impact body on the excitation surface. For example, the impact body and/or the elastic element can be made of stainless steel, titanium and/or amorphous metallic glass. Amorphous metallic glass and in particular solid metallic glass are particularly harder than their crystalline metal compounds and have a high strength. In addition, small deformations in the single-digit percentage range are purely elastic, so that the energy absorbed is not lost as deformation energy, but is completely released again when the metallic glass springs back.

“Mechanical impact excitation” is understood to mean in particular that the sonotrode is excited to vibrate by striking the impact body. The mechanical impact excitation is in particular a discontinuous, intermittent and/or oscillating impact wave and/or shock wave. The vibration excited by the mechanical impact excitation has in particular a greater intensity and/or amplitude and/or a time-varying wavelength compared to the vibration excitation by means of the vibration excitation device. Due to the oblique impact of the at least one impact body on the excitation surface and thus the mechanical impact excitation, in particular, a radial vibration of the sonotrode is excited. A “radial vibration” is understood to mean, in particular, a vibration in the direction of the radius of the sonotrode. A radial vibration can also be directed outwards from the center point and thus the longitudinal center axis of the sonotrode.

An “excitation surface” is in particular a surface and therefore an area or region that extends over a length and a width. The excitation surface is in particular the surface of an element, component and/or assembly of the lithotripsy device on which one or more impact bodies strike. The excitation surface lies in the direction of the longitudinal center axis of the sonotrode, in particular not within a cross-section of the sonotrode. In particular, the excitation surface is not arranged coaxially to the sonotrode. In particular, the excitation surface can have a larger area than the striking surface and thus the contact point and/or contact region of one or more impact bodies. The excitation surface is not permanently installed, in particular in the direction of the longitudinal center axis of the sonotrode. Preferably, the excitation surface and/or an element or component with the excitation surface is predominantly present with a free surface and is only connected directly or indirectly to the sonotrode and/or the horn on one side, for example. In particular, the excitation surface itself can be excited to vibrate. In particular, the excitation surface has a special shape, material constrictions and/or cut-outs.

In a further embodiment, the lithotripsy device has a second impact body, a third impact body, a fourth impact body and/or further impact bodies, wherein a respective clamped elastic element is arranged at one end of each impact body and the respective impact body can be struck obliquely with its free end onto the first excitation surface.

As a result of the fact that an impact body is not arranged coaxially to the longitudinal center axis of the sonotrode, but strikes the excitation surface obliquely, a plurality of impacting bodies can be easily arranged so that they strike the first excitation surface obliquely. Thus, for example, a plurality of impact bodies can be arranged freely around the sonotrode at an oblique radial angle and/or evenly spaced.

The function of a second, third, fourth and/or further impact body is the same as that of the impact body defined above, but the impact bodies can in each case have the same or different properties. Thus, different striking masses, different shapes and/or materials can be used for the individual impact bodies, thereby specifically influencing the radial vibration of the sonotrode. Consequently, the impact bodies can in particular have different striking directions, striking forces and/or striking frequencies.

In order to utilize a different oscillation of the respective spring-mass system, different striking directions and/or a uniform or non-uniform distribution of the striking surfaces around the longitudinal center axis of the sonotrode and/or the horn, the lithotripsy device has a second excitation surface, a third excitation surface and/or further excitation surfaces, so that the second impact body, the third impact body, the fourth impact body and/or the further impact bodies can hit the second excitation surface, the third excitation surface and/or the further excitation surfaces obliquely.

In principle, it should be emphasized that in each case only a single impact body can strike an excitation surface or any number of impact bodies can act on an excitation surface. Thus, both the number of impact bodies acting on each excitation surface and the number of excitation surfaces can be freely selected. The plurality of excitation surfaces can also form different segments of an entire striking surface and/or a component, for example on a striking body. The individual excitation surfaces can also be connected in each case directly or indirectly in one piece to the horn and/or sonotrode. Above all, the plurality of excitation surfaces can vibrate differently when excited by vibration, in particular ultrasonic vibration excitation, and thus repulse the mass-spring system of the impact body connected to the elastic element in different manners and strengths.

In a further embodiment of the lithotripsy device, the first excitation surface, the respective excitation surface and/or the excitation surfaces are designed to be arranged or curved obliquely to the longitudinal center axis of the sonotrode and to the transverse axis of the sonotrode.

Due to this oblique position of the respective excitation surface in relation to the longitudinal center axis of the sonotrode and/or a curved design of the respective excitation surface, the radial vibration of the sonotrode upon the hitting of the respective impact body is increased. Due to this oblique position of the respective excitation surface, the impact body hits the excitation surface with its free end from the oblique hitting direction over the entire surface and not just with one of its edges. Thus, the degree of radial excitation of the sonotrode can be specifically influenced and defined both by the oblique hitting direction of the respective impact body and by the oblique position of the respective excitation surface.

With a curved design of the excitation surface or excitation surfaces, a differently pronounced bend or the same bend can also be formed. Preferably, the excitation surface or the excitation surfaces have a convex surface opposite to the striking direction. Thus, the excitation surface or the excitation surfaces are curved inwards, in particular in the direction of the distal end of the sonotrode.

In order to realize a defined alignment of the respective excitation surface and/or identical or different oblique positions of the excitation surfaces, the first excitation surface, the respective excitation surface and/or the excitation surfaces have the same angle or a different angle to the longitudinal center axis of the sonotrode.

In a further embodiment, the first excitation surface, the respective excitation surface and/or the excitation surfaces have the same material thickness and/or a different material thickness in the longitudinal direction.

Due to the selection of the respective material thickness in the longitudinal direction of the sonotrode and/or the respective impact body, the vibration behavior of each excitation surface can be specifically influenced and/or configured differently. Thus, each excitation surface can be divided into a plurality of sections with different and/or inhomogeneous thicknesses. The plurality of excitation surfaces can also have different thicknesses in relation to one another and/or inhomogeneous thickness profiles over the respective surface in the direction of the longitudinal direction of the sonotrode and/or the longitudinal direction of the respective impact body.

In order to achieve a vibration of the respective excitation surface at a predetermined frequency upon impact of the respective impact body, the excitation surface, the respective excitation surface and/or the excitation surfaces are designed as flexural resonators.

Thus, the respective excitation surface acts as a tuning fork. For this purpose, for example, the respective excitation surface can have corresponding material constrictions and/or cut-outs.

A “flexural resonator” is, in particular, a spring-mass system capable of harmonic vibration. The respective excitation surface is designed in particular in the form of a flexural resonator. If the respective excitation surface is connected directly or indirectly on one side around the sonotrode and/or the horn, the bending moments and masses distributed along the radial direction of the excitation surface determine the natural frequency of the excitation surface as a flexural resonator. Thus, the respective excitation surface can be designed as a tuning fork. In particular in the case of a striking body with a plurality of excitation surfaces designed as flexural resonators, the excitation surfaces can vibrate in opposite directions and/or in different directions when the respective impact body strikes. In particular, the plurality of excitation surfaces are in each case shaped in such a way that they can vibrate like a tuning fork and thus induce a specific frequency.

In a further embodiment of the lithotripsy device, the excitation surface, the respective excitation surface and/or the excitation surfaces is or are arranged on the sonotrode or is or are connected in one piece to the sonotrode.

When the respective excitation surface is arranged on the sonotrode and/or the horn, the excitation surfaces are individually or jointly connected to the sonotrode and/or the horn, in particular in a detachable manner. “One piece” is understood to mean that the respective excitation surface is connected to the horn and/or sonotrode in a manner that is permanent and thus not detachable. Advantageously, this is understood to mean that the respective excitation surface and the sonotrode and/or the horn are formed from one piece and/or are produced in a state connected together.

In order to specifically influence the radial vibration of the sonotrode along its circumference, the respective longitudinal direction of the first impact body, the respective impact body and/or the impact bodies has or have the same angle and/or a different angle to the longitudinal center axis of the sonotrode.

Due to the different or same angles and thus oblique positions of the respective impact bodies, a uniform drilling effect around the sonotrode tip and/or a specific uneven drilling effect around the circumference of the sonotrode tip can be adjusted.

In a further embodiment of the lithotripsy device, the first impact body, the respective impact body and/or the impact bodies have the same mass and/or a different mass.

As a result, both a different inertia of the mass-spring system of the impact body with the respective connected elastic element and a strength of the impact on the respective excitation surface can be adjusted.

In order to specifically adjust the excitation of the oscillating back and forth movement of the respective impact body in the striking direction and against the striking direction, the clamped elastic element, the respective clamped elastic elements and/or the clamped elastic elements have the same elasticity tension and/or a different elasticity tension.

Due to the variation of the aforementioned parameters and properties of the respective impact bodies, the respective excitation surfaces and/or the respective clamped elastic elements, the mechanical impact excitation and thus the radial vibration of the sonotrode can be influenced in a variety of manners. Since a plurality of striking masses and spring combinations can be realized in the lithotripsy device, these can be varied in many ways to achieve a preferred striking pattern. Thus, for example, the masses of the striking bodies, the spring properties of the elastic elements and/or their pretensioning along with the oblique position and/or the vibration behavior of the excitation surfaces can be varied in a specific manner. Thus, a defined, intensive radial vibration of the sonotrode can be achieved.

In a further embodiment of the lithotripsy device, the vibration excitation device comprises an ultrasonic vibration exciter and/or the lithotripsy device comprises a horn having a distal end and a proximal end, wherein the sonotrode is connectable to the distal end or the proximal end of the horn and the ultrasonic vibration exciter is electrically connectable to an assigned ultrasonic generator, so that the sonotrode can be excited to a longitudinal vibration by means of the ultrasonic vibration exciter.

In addition to the usually continuous, longitudinal vibration excitation of the sonotrode by means of ultrasound, the respective mass-spring system consisting of impact body and elastic element and the respective excitation surface are also excited to vibrate by means of the ultrasonic vibration exciter. As a result of the fact that the impact body is pressed obliquely by the spring onto the ultrasonically vibrating excitation surface, the impact body is repulsed directly by means of the ultrasonic vibration of the excitation surface and the spring is tensioned.

Consequently, as a result of the two excitations of the excitation surface and the impact body, the generated ultrasonic vibration causes an oscillating back and forth movement of the spring-mass system and enables improved repulsion.

In order to specifically arrange an excitation surface or a plurality of excitation surfaces on a sonotrode and/or the horn, the lithotripsy device has at least one striking body, wherein the at least one striking body has the excitation surface, the respective excitation surface and/or the excitation surfaces and is connectable to the sonotrode and/or the horn.

Thus, the striking body can be arranged at a defined position in the direction of the longitudinal center axis on the sonotrode and/or the horn. The striking body can also be used to retrofit an existing sonotrode and/or horn.

A “striking body” is understood to mean in particular a body, a component and/or a component group that has at least one excitation surface or a plurality of excitation surfaces. In particular, the striking body has a connecting element for direct or indirect connection to the sonotrode and/or the horn. Of course, a plurality of striking bodies can also be arranged on the sonotrode and/or the horn. A striking body can also be arranged at the transition between the sonotrode head and the distal end of the horn and thus be connected to both.

In order to arrange the striking body around the outer surface and thus along the outer diameter of the sonotrode and/or the horn, the at least one striking body has a through hole in one direction of the longitudinal center axis of the sonotrode for sliding onto the sonotrode and/or the horn.

Thus, the striking body with the through hole can be easily retrofitted to an existing sonotrode and/or horn.

In principle, it should be emphasized that the through hole can be arranged in the middle of the cross-section of the striking body, but can also be made eccentrically through the striking body.

In order to locally adjust and/or amplify the radial vibration of the sonotrode and/or additionally arrange a striking body on the horn, the lithotripsy device has a second striking body and/or further striking bodies, in each case having an assigned impact body with a clamped elastic element and/or having a plurality of assigned impact bodies in each case with a clamped elastic element.

A striking body on the sonotrode and a striking body on the horn can be configured differently. Preferably, the striking body can also be arranged on a rearwardly aligned horn that, contrary to the usual design, tapers in the proximal direction, wherein the vibrations transmitted by the horn are reflected at a proximal stop surface and transmitted to the proximal end of the sonotrode. In this case, a radial vibration of the proximal end of the sonotrode is induced, which is transmitted along the longitudinal center axis of the sonotrode to the distal tip of the sonotrode.

In a further aspect of the invention, the object is achieved by an impact body for mechanical impact excitation of a sonotrode, wherein the impact body has a longitudinal direction, a proximal end and a distal end and is connected at its proximal end or at its distal end to a clampable elastic element, so that the impact body has a free end, wherein the impact body and the clampable elastic element are designed in such a way that the impact body can be excited to vibrate by means of a vibration excitation device through an ultrasonic vibration, so that the free end of the impact body can be struck obliquely against a first excitation surface for mechanical impact excitation of the sonotrode.

Advantageously, the impact body can be designed as a mass in such a way that the ultrasonic vibration is a harmonic of the mass-spring system. Efficiency can also be increased if the mass-spring system has a natural vibration and/or natural frequency that corresponds to a natural frequency, in particular a low natural frequency, of the ultrasonic system.

In an additional aspect of the invention, the object is achieved by a retrofit kit for retrofitting an existing lithotripsy device, wherein the existing lithotripsy device comprises a sonotrode and/or a horn, wherein the retrofit kit comprises at least one striking body with one or more excitation surfaces and at least one impact body connected to a clampable elastic element at its one end or a plurality of impact bodies in each case connected to a clampable elastic element at one end of the respective impact body, and the striking body comprises at least one adapter for mechanical connection to the sonotrode and/or the horn, so that a lithotripsy device as described above can be formed.

It is particularly advantageous that the retrofit kit can also be used to retrofit sonotrodes and/or lithotripsy devices already on the market in order to specifically excite a radial vibration of the sonotrode. A receptacle and/or holder for the elastic element can also be retrofitted at the opposite end to the impact body. Preferably, the retrofit kit has a plurality of adapters so that the respective striking body can be connected to different sonotrodes and/or horns, in particular with their different outer diameters.

In a further aspect, the object is achieved by a method for operating a lithotripsy device, wherein the lithotripsy device has a sonotrode, at least one impact body for mechanical impact excitation of the sonotrode, at least one excitation surface and a vibration excitation device for vibration excitation of the sonotrode and the at least one impact body, wherein the at least one impact body is connected with its one end to a clamped elastic element and is directed with its free end towards the first excitation surface, and the at least one impact body is arranged with its longitudinal direction deviating from a longitudinal center axis of the sonotrode and deviating from a transverse axis of the sonotrode, the method having the following steps:
Excitation of a first vibration, in particular a longitudinal vibration, of the sonotrode by means of the vibration excitation device,
Excitation of a vibration of the at least one impact body by means of the vibration excitation device and movement of the free end of the at least one impact body against the first excitation surface by means of the elastic element, and
Excitation of a radial vibration of the sonotrode by striking the free end of the at least one impact body against the first excitation surface.

In principle, these method steps can be carried out and/or repeated simultaneously or alternately in any order. When the method steps are carried out simultaneously, there is a combined operation and a dual effect by means of the continuous vibration excitation by means of the vibration excitation device and the radial vibration of the sonotrode by means of the mechanical impact excitation.

Thus, by means of the method, the user can induce and use a radial vibration in addition to a preferably longitudinal vibration of the sonotrode in a very simple and specific manner. In principle, it should be emphasized that the claimed method steps concern the operation and generation of two different vibration modes within a lithotripsy device and that the method for operating the lithotripsy device does not comprise a treatment step and thus does not constitute a treatment method. The method can also be used for trial operation in particular.

Optionally, as a further method step, an excitation of a vibration of the excitation surface takes place simultaneously or additionally. As a result, after the striking of the free end of the at least one impact body, the impact body is repulsed more strongly and the spring is better tensioned.

The invention is explained in more detail below with reference to exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a highly schematic three-dimensional representation of a conventional lithotripsy system with a lithotripsy device and an ultrasound generator and a supply and control unit;

FIG. 2 is a highly schematic representation of a retrofitted lithotripsy device according to the invention with an adapter piece and two striking masses on a sonotrode;

FIG. 3 is highly schematic representation of a lithotripsy device with an alternative of the adapter piece designed as a flexural resonator;

FIG. 4 is a schematic representation of the top view of the adapter piece in a transmission direction;

FIG. 5 is a highly schematic representation of an alternative of the lithotripsy device with a rearward-facing horn and an alternative of the adapter piece arranged around the horn; and

FIG. 6 is a schematic representation of method steps of a method for operating a lithotripsy device.

### DESCRIPTION OF PREFERRED EMBODIMENTS

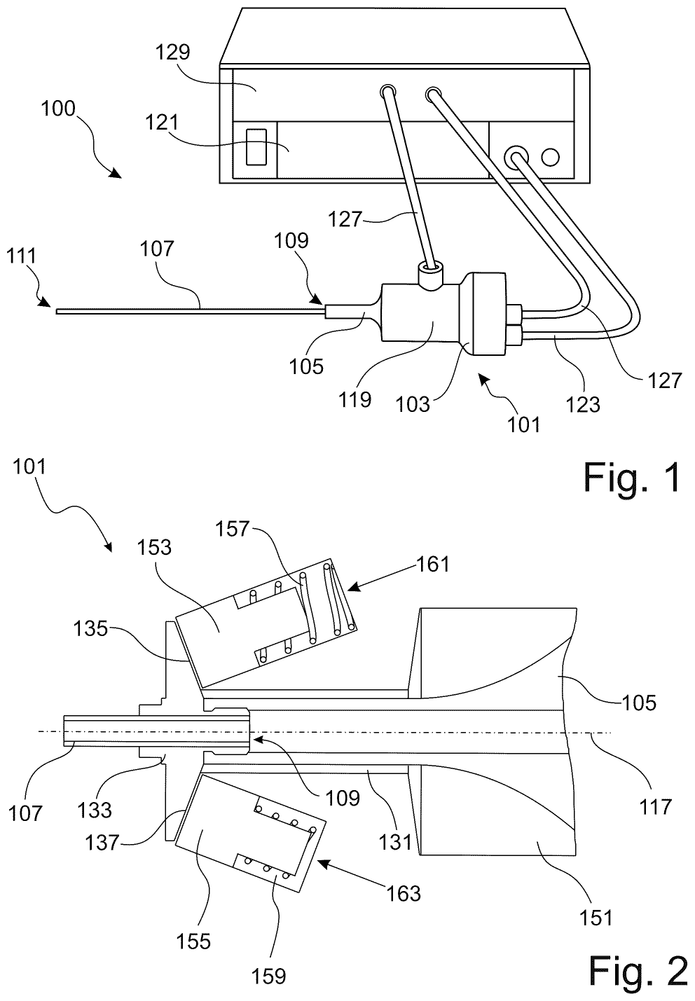

Referring to the drawings, a conventional lithotripsy system 100 has a lithotripsy device 101, an ultrasound generator 121 and a supply and control unit 129. The lithotripsy device 101 has an ultrasonic transducer 103 for generating an ultrasonic vibration and a handle 119. A tapered horn 105 is arranged on the distal side of the ultrasonic transducer 103 in a transmission direction 165. A sonotrode 107 is screwed into the horn 105 at its proximal end 109. The lithotripsy device 101 is designed as an intracorporeal lithotripter, wherein a distal end 111 of the sonotrode 107 serves to act directly on and fragment body stones. The ultrasonic transducer 103 serves to excite the sonotrode 107 with a continuous ultrasonic vibration, wherein the ultrasonic waves are transmitted from the ultrasonic transducer 103 by means of the horn 105 to the sonotrode head at the proximal end 109 of the sonotrode 107 and are further guided by the sonotrode 107 to its distal end 111. The ultrasonic transducer 103 is electrically connected to the ultrasonic generator 121 by means of a connecting cable 123. Furthermore, the lithotripsy device 101 is connected to two hoses 127 for media supply and/or for rinsing (FIG. 1).

The conventional lithotripsy device shown in FIG. 1 has been retrofitted by means of an adapter piece 133 and two spring-mass systems, in each case consisting of a first striking mass 153 and a first spring 157 and a second striking mass 155 and a second spring 159. For this purpose, the adapter piece 133 is inserted on the proximal side into the horn 105 and screwed in. The sonotrode 107 is designed as a long tube and a proximal section of the sonotrode 107 is received continuously along a longitudinal center axis 117 of the sonotrode 107 in the adapter piece 133 on the inside, so that the proximal end 109 of the sonotrode 107 rests against the horn 105.

The adapter piece 133 has a rotationally symmetrical, obliquely positioned curved surface on the proximal side, with a first striking surface 135 for the first striking mass 153 in a first striking direction 161 and a second striking surface 137 for the second striking mass 155 in a second striking direction 163. The first striking mass 153 and the second striking mass 155 in each case engage on the proximal side in coils of the first spring 157 and the second spring 159 and form a permanent connection. The respective proximal-side end of the first spring 157 and the second spring 159 are received in corresponding receptacles (not shown in FIG. 2) and are limited in their extension, wherein the receptacles (not shown) are fastened to an attachment 151 on the horn 105. On the distal side, the horn 105 is encompassed by a holder 131, which also surrounds the adjacent adapter piece 133, as a result of which the ability to clean is improved.

The retrofitted lithotripsy device 101 according to the invention shown in FIG. 2 is used to carry out the following operations:

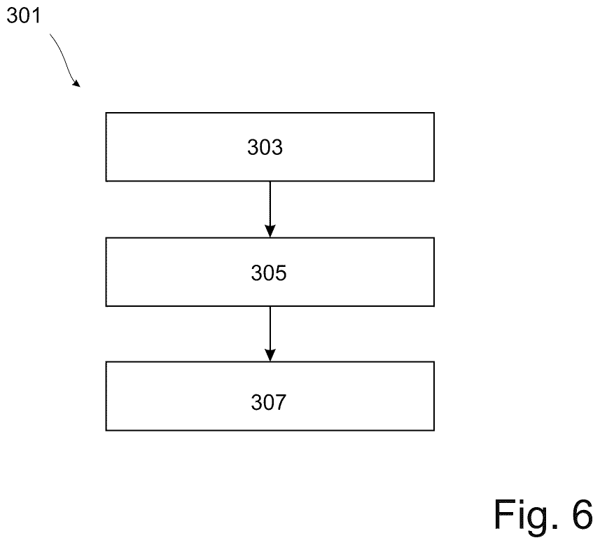

In a method 301 (FIG. 6) for operating the lithotripsy device 101, an excitation 303 of a first longitudinal vibration of the sonotrode 107 is effected by means of an ultrasonic transducer 103 (as described with respect to FIG. 1), in which the ultrasonic generator 121 applies a voltage to the ultrasonic transducer 103. As a result of a deformation of piezo elements within the ultrasonic transducer 103, an ultrasonic vibration is induced, as a result of which the excitation 303 of the first longitudinal vibration of the sonotrode 107 is effected. At the same time, due to the generated ultrasonic vibration, an excitation 305 of a vibration of the first striking mass 153 and the second striking mass 155 is effected, and a movement of the respective distal end of the first striking mass 153 and the second striking mass 155 is effected in the first striking direction 161 and the second striking direction 163 against the first striking surface 135 and the second striking surface 137 (FIG. 2 shows the state upon striking). Due to the oblique impact of the first striking mass 153 and the second striking mass 155 on the curved first and second striking surfaces 135, 137, an excitation 307 of a radial vibration of the sonotrode 107 is effected, as a result of which a drilling effect in the radial direction can be utilized at the distal end 111 of the sonotrode tip in addition to the penetration of the sonotrode tip in the axial direction.

As a result of the shape of the adapter piece 133 with the first striking surface 135 and the second striking surface 137 with its bend, these striking surfaces 135 and 137 are also excited to vibrate by means of the ultrasonic vibration of the ultrasonic transducer 103 itself and thereby repulse the first striking mass 153 and the second striking mass 155 against the first striking direction 161 and the second striking direction 163, as a result of which the first spring 157 and the second spring 159 are tensioned again and the operations are repeated.

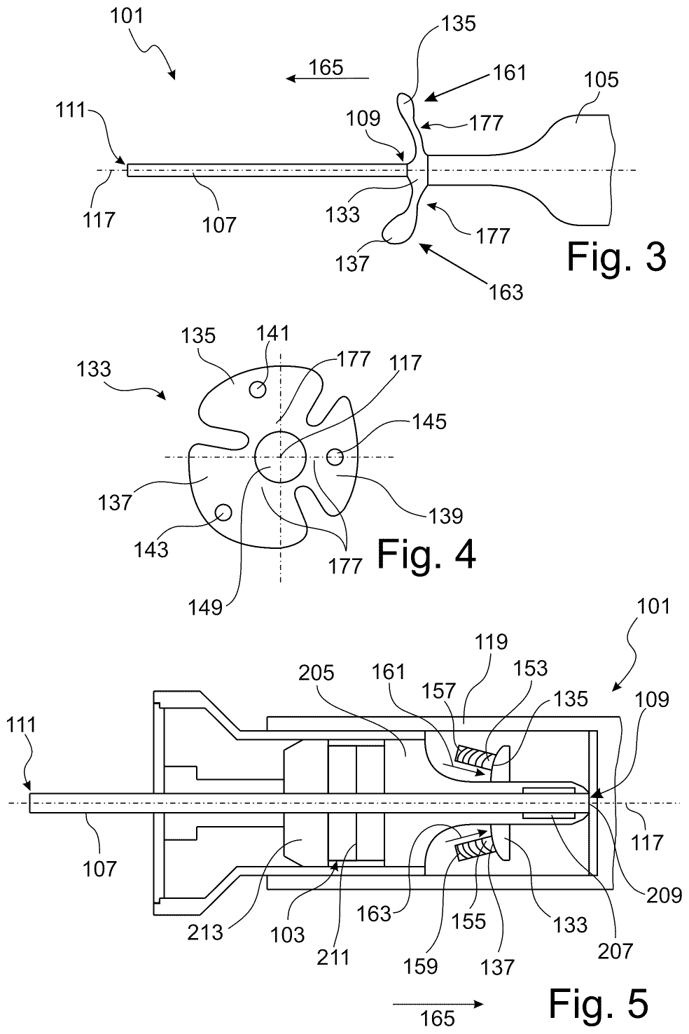

In one alternative of the lithotripsy device 101, an alternative adapter piece 133 is designed as a flexural resonator and is arranged at the transition between a sonotrode 107 and a horn 105. The adapter piece 133 has a hole 149, which is arranged centrally around the longitudinal center axis 117 of the sonotrode 107 on the outside of the sonotrode 107 and adjacent to the horn 107. The adapter piece 133 has a first striking surface 135 with a first striking point 141, a second striking surface 137 with a second striking point 143 and a third striking surface 139 with a third striking point 145. Radially outwardly, each striking surface 135, 137 and 139 has a constriction 177 with a smaller material thickness in a transmission direction 165 and subsequently expands its respective shape. As a result, each of the striking surfaces 135, 137 and 139 is designed as a flexural resonator and thus as a tuning fork with a specific frequency. Three associated striking masses with respective springs (not shown in FIG. 3) are held and clamped in corresponding receptacles on the horn 105, as described with respect to FIG. 2, and in each case impact obliquely in a first striking direction 161 and a second striking direction 163 (the third striking direction is not visible in FIG. 3) on the respective striking points 141, 143 and 145. As a result of the uneven arrangement of the striking points 141, 143 and 145 on the adapter piece 133 and as a result of the uneven shaping and constrictions 177 of the three striking surfaces 135, 137 and 139, the induced radial vibration of the sonotrode 107 is configured differently in a radially circumferential manner.

In an alternative, a lithotripsy device 101 has an ultrasonic transducer 103 with piezo elements 211, which are clamped between a counter bearing 213 and a horn 205. The ultrasonic transducer 103, the horn 205 and a proximal-side stop surface 209 are surrounded by a handle 119 as a housing. In contrast to the embodiment shown in FIG. 1, the lithotripsy device 101 has the rearward-facing horn 205, as a result of which a transmission direction 165 is directed from the ultrasonic transducer 103 to the proximal end of the horn 205. With this design, the sonotrode 107 is guided on the inside through the entire length of the rearward-facing horn 205 and rests with its proximal end 109 against the stop surface 209, wherein the sonotrode 107 is mounted in its proximal section in the horn 205 by means of a bearing 207.

An adapter piece 133 with a first curved striking surface 135 and a second curved striking surface 137, which are curved against the transmission direction 165, is aligned on the distal side of the bearing 207. As described above, receptacles for two mass-spring systems consisting of a first striking mass 153 with a connected first spring 157 and a second striking mass 155 connected to a second spring 159 are formed on the rearward-facing horn 205 on a holder (not shown). Thus, the two spring-mass systems are directed obliquely in a first striking direction 161 and a second striking direction 163 onto the two striking surfaces 135 and 137. The ultrasonic vibration generated by the ultrasonic transducer 103 by means of the piezo elements 211 is directed to the proximal end 109 of the sonotrode 107 and focused due to the rearward-facing horn 205, and at the same time the first striking mass 153 and the second striking mass 155 along with the first striking surface 135 and the second striking surface 137 are excited to vibrate by means of the ultrasonic vibration and thereby the first striking mass 153 and the second striking mass 155 are pressed in an oscillating manner by means of the respective spring 157 and 159 against the first striking surface 135 and the second striking surface 137 and are repulsed as a result of the vibration of these two striking surfaces 135 and 137, as a result of which a radial vibration excitation is transferred in an oscillating manner to the proximal end of the sonotrode 107 and reflected at the stop surface 109 and forwarded via the sonotrode 107 to its distal end 111, as a result of which a radial drilling effect at the distal end of the sonotrode can be used to fragment body stones.

Thus, different variants of a lithotripsy device 101 are provided, with which a defined, reliable and intensive radial vibration of the sonotrode 107 can be generated by means of one or more oscillating, obliquely hitting striking masses 153, 155.

The invention relates to a lithotripsy device, in particular an intracorporeal lithotripsy device, for fragmenting body stones, wherein the lithotripsy device has a carrier unit, a sonotrode that is connectable to the carrier unit at the distal end and has a longitudinal center axis, and at least one impact body for mechanical impact excitation of the sonotrode, wherein the at least one impact body has a longitudinal direction, a proximal end and a distal end and is connected at its proximal end or at its distal end to a clamped elastic element, so that the at least one impact body has a free end, and the lithotripsy device has a vibration excitation device for vibration excitation of the sonotrode and of the at least one impact body, and the free end of the at least one impact body is directed towards a first excitation surface and can be pressed against the first excitation surface by means of the clamped elastic element upon vibration excitation, wherein the excitation surface is directly or indirectly connected to the sonotrode, wherein the at least one impact body is arranged with its longitudinal direction in a direction deviating from the longitudinal center axis of the sonotrode and deviating from a transverse axis of the sonotrode, so that radial vibration of the sonotrode can be excited by an oblique strike of the at least one impact body with its free end onto the first excitation surface. Furthermore, the invention relates to an impact body, a retrofit kit and a method for operating a lithotripsy device.

While specific embodiments of the invention have been shown and described in detail to illustrate the application of the principles of the invention, it will be understood that the invention may be embodied otherwise without departing from such principles.

### LIST OF REFERENCE SIGNS

100 Lithotripsy system
101 Lithotripsy device
103 Ultrasonic transducer
105 Horn
107 Sonotrode
109 Proximal end of the sonotrode
111 Distal end of the sonotrode
117 Longitudinal center axis of the sonotrode
119 Handle
121 Ultrasonic generator
123 Connecting cable
127 Hose
129 Supply and control unit
131 Holder
133 Adapter piece
135 First striking surface
137 Second striking surface
139 Third striking surface
141 First striking point
143 Second striking point
145 Third striking point
149 Hole
151 Attachment
153 First striking mass
155 Second striking mass
157 First spring
159 Second spring
161 First striking direction
163 Second striking direction
165 Transmission direction
171 First vibration segment
173 Second vibration segment
175 Third vibration segment
177 Constriction
205 Rearward-facing horn
207 Bearing
209 Stop surface
211 Piezo elements
213 Counter bearing
301 Method for operating a lithotripsy device
303 Excitation of a first vibration of the sonotrode
305 Excitation of a vibration of an impact body and movement of the impact body against an excitation surface
307 Excitation of a radial vibration of the sonotrode

1. A lithotripsy device for fragmenting body stones, the lithotripsy device has comprising:
a carrier unit;
a sonotrode that is connectable to the carrier unit on the distal side and has a longitudinal center axis;
at least one impact body for mechanical impact excitation of the sonotrode, wherein the at least one impact body has a longitudinal direction, a proximal end and a distal end and is connected at the proximal end or at the distal end of the at least one impact body to a clamped elastic element, so that the at least one impact body has a free end; and
a vibration excitation device for vibration excitation of the sonotrode and of the at least one impact body, and wherein the free end of the at least one impact body is directed towards a first excitation surface and is configured to be pressed against the first excitation surface by means of the clamped elastic element upon vibration excitation, wherein the excitation surface is connected directly or indirectly to the sonotrode, wherein the at least one impact body is arranged with a longitudinal direction of the least one impact body in a direction deviating from the longitudinal center axis of the sonotrode and deviating from a transverse axis of the sonotrode, so that a radial vibration of the sonotrode is configured to be excited by an oblique strike of a free end of the at least one impact body onto the first excitation surface.

2. The lithotripsy device according to claim 1, wherein the lithotripsy device further comprises a second impact body, a third impact body, a fourth impact body and/or further impact bodies, wherein a respective clamped elastic element is arranged at one end of each impact body and the respective impact body is configured to strike obliquely with a free end of the respective impact body onto the first excitation surface.

3. The lithotripsy device according to claim 1, wherein the lithotripsy device further comprises a second impact body, a third impact body, a fourth impact body and/or further impact bodies, wherein a respective clamped elastic element is arranged at one end of each impact body, and a second excitation surface, a third excitation surface and/or further excitation surfaces, so that the second impact body, the third impact body, the fourth impact body and/or the further impact bodies are configured to hit the second excitation surface, the third excitation surface and/or the further excitation surfaces obliquely.

4. The lithotripsy device according to claim 1, wherein the first excitation surface is configured to be arranged or curved obliquely to the longitudinal center axis of the sonotrode and to the transverse axis of the sonotrode.

5. The lithotripsy device according to claim 3, wherein the first excitation surface, the respective excitation surface and/or the excitation surfaces have the same angle and/or a different angle to the longitudinal center axis of the sonotrode.

6. The lithotripsy device according to claim 3, wherein the first excitation surface, the respective excitation surface and/or the excitation surfaces have the same material thickness and/or a different material thickness in the longitudinal direction.

7. The lithotripsy device according to claim 1, wherein the excitation surface, configured as a flexural resonator.

8. The lithotripsy device according to claim 1, wherein the excitation surface is arranged on the sonotrode or is connected in one piece to the sonotrode.

9. The lithotripsy device according to claim 2, wherein the respective longitudinal direction of the first impact body, the respective impact body and/or the impact bodies has or have the same angle and/or different angles to the longitudinal center axis of the sonotrode.

10. The lithotripsy device according to claim 2, wherein the first impact body, the respective impact body and/or the impact bodies has or have the same mass and/or a different mass.

11. The lithotripsy device according to claim 2, wherein the clamped elastic element, the respective clamped elastic elements and/or the clamped elastic elements has or have the same elasticity tension and/or a different elasticity tension.

12. The lithotripsy device according to claim 1, wherein the vibration excitation device comprises an ultrasonic vibration exciter and the lithotripsy device further comprises a horn having a distal end and a proximal end, wherein the sonotrode is connectable to the distal end or the proximal end of the horn and the ultrasonic vibration exciter is electrically connectable to an assignable ultrasonic generator, so that the sonotrode is configured to be excited to a longitudinal vibration by means of the ultrasonic vibration exciter.

13. The lithotripsy device according to claim 1, further comprising at least one striking body, wherein the at least one striking body comprises the excitation surface, the respective excitation surface and/or the excitation surfaces and is connectable to the sonotrode and/or the horn.

14. The lithotripsy device according to claim 13, wherein the at least one striking body has a through hole in a direction of the longitudinal center axis of the sonotrode for sliding onto the sonotrode and/or the horn.

15. The lithotripsy device according to claim 13, further comprising a second striking body and/or further striking bodies, in each case having an assigned impact body with a clamped elastic element or having a plurality of assigned impact bodies in each case with a clamped elastic element.

16. An impact body for mechanical impact excitation of a sonotrode, wherein the impact body comprises a longitudinal direction, a proximal end and a distal end and is connected at the proximal end or at the distal end to a clampable elastic element, so that the impact body has a free end, wherein the impact body and the clampable elastic element are configured in such a way that the impact body can be is excited to vibrate by means of a vibration excitation device through an ultrasonic vibration, so that the free end of the impact body is configured to strike obliquely against a first excitation surface for mechanical impact excitation of the sonotrode.

17. A retrofit kit for retrofitting an existing lithotripsy device, wherein the existing lithotripsy device comprises a sonotrode and/or a horn, the retrofit kit comprising:
at least one striking body with one excitation surface or a plurality of excitation surfaces; and
at least one impact body connected to a clampable elastic element at an impact body end or a plurality of impact bodies in each case connected to a clampable elastic element at one end of the respective impact body, and wherein the striking body comprises at least one adapter for mechanical connection to the sonotrode and/or the horn, to form a lithotripsy device comprising a carrier unit; the sonotrode that is connectable to the carrier unit on the distal side and has a longitudinal center axis; and the at least one impact body for mechanical impact excitation of the sonotrode, wherein the at least one impact body has a longitudinal direction, a proximal end, and a distal end, wherein the distal end is connected at the proximal end or at the distal end to the clamped elastic element, so that the at least one impact body has a free end, and the lithotripsy device has a vibration excitation device for vibration excitation of the sonotrode and of the at least one impact body, and the free end of the at least one impact body is directed towards a first excitation surface and is configured to be pressed against the first excitation surface by means of the clamped elastic element upon vibration excitation, wherein the excitation surface is connected directly or indirectly to the sonotrode, wherein the at least one impact body is arranged with its longitudinal direction in a direction deviating from the longitudinal center axis of the sonotrode and deviating from a transverse axis of the sonotrode, so that a radial vibration of the sonotrode can be excited by an oblique strike of the at least one impact body with its free end onto the first excitation surface.

18. A method for operating a lithotripsy device, wherein the lithotripsy device comprises: a sonotrode; at least one impact body for mechanical impact excitation of the sonotrode, at least one excitation surface; and a vibration excitation device for vibration excitation of the sonotrode and of the at least one impact body, wherein the at least one impact body is connected with its one end to a clamped elastic element and is directed with its free end towards the first excitation surface, and the at least one impact body is arranged with its longitudinal direction deviating from a longitudinal center axis of the sonotrode and deviating from a transverse axis of the sonotrode, the method comprising the steps of:
exciting a first vibration, in particular a longitudinal vibration, of the sonotrode by means of the vibration excitation device,
exciting a vibration of the at least one impact body by means of the vibration excitation device and movement of the free end of the at least one impact body against the first excitation surface by means of the elastic element, and
exciting a radial vibration of the sonotrode by striking the free end of the at least one impact body against the first excitation surface.
